(19) United States
(12) Patent Application Publication
(10) Pub. No.: US 2009/0028841 A1
(43) Pub. Date: Jan. 29, 2009

(54) OXYGEN SCAVENGING COMPOSITION, COATING COMPOSITION AND PACKAGE CONTAINING FREE FATTY ACID

(76) Inventor: RAMESHCHANDRA M. GOHIL, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/177,422

(22) Filed: Jul. 22, 2008

Related U.S. Application Data
(60) Provisional application No. 60/951,723, filed on Jul. 25, 2007.
(60) Provisional application No. 60/951,720, filed on Jul. 25, 2007.
(60) Provisional application No. 60/951,722, filed on Jul. 25, 2007.

(51) Int. Cl. A61K 38/44; C12N 11/00; A23L 1/48; C09K 15/20; C09K 15/06; C12N 9/02

(52) U.S. Cl. 424/94.4; 435/174; 435/189; 426/61; 252/407; 252/398; 252/403

(57) ABSTRACT

An oxygen scavenging composition being an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative. The invention extends to a coating composition thereof and a package or container containing the oxygen scavenging composition.

### FIELD OF THE INVENTION

The present invention is directed to improved oxygen-scavenging compositions and coating compositions for use in oxygen-reduced packaging.

### BACKGROUND OF THE INVENTION

Farneth et al., United States Patent Publication 2005-020584, disclose compositions of calcium ascorbate and laccase enzyme as effective oxygen scavenging compositions for use in reduced-oxygen packaging.

There remains a need in the industry for an improved oxygen scavenging composition for use in preserving oxygen sensitive consumer products.

### SUMMARY OF THE INVENTION

The present invention provides a composition comprising an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative.

The present invention further provides a method comprising exposing to oxygen in the presence of moisture a composition comprising an oxidizable ascorbic acid derivative, a multi-copper oxidase enzyme, and free fatty acid wherein the enzyme is disposed upon the surface of the oxidizable ascorbic acid derivative, and wherein the freee fatty acid is intermixed with the oxidizable ascorbic acid derivative The present invention, also, provides a coating composition containing the composition found above dissolved or suspended in a carrrier; and a package having such composition therein.

### BRIEF DESCRIPTION OF THE FIGURES

FIG. 1 is a schematic diagram showing a multi-layer structure comprising the oxygen scavenging composition of the invention.

### DETAILED DESCRIPTION

The present invention is useful for preserving a variety of oxygen-sensitive electronic components, inerting aircraft fuel tanks, preserving oxygen sensitive pharmaceutical compositions, generating and maintaining an oxygen-free atmosphere for culturing anaerobic microorganisms, preserving cosmetics and personal care products, and preservation of package or containerd foodstuffs and beverages.

Advantages incurred by use of the O₂ scavenging systems of the invention herein include: food-safe components, easily applied water-based formulations, and the ability to apply the oxygen-scavenging composition in thin layers.

The present invention is directed to an oxygen-scavenging composition, a process for removing oxygen (O₂) from a sealed container, a coated article suitable for use in package or container goods, and a package or container.

Accordingly a composition is provided comprising an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative.

Further provided is a process comprising contacting the composition with molecular oxygen in the presence of moisture thereby causing at least a portion of the oxygen to be reduced by reaction with the oxidizable ascorbic acid derivative.

For the purposes of the present invention the term “free fatty acid” is used to refer to an unsaturated fatty acid that is not chemically bound to the ascorbic acid moiety but is rather physically mixed therewith. In one embodiment of the invention, the oxidizable ascorbic acid derivative is an ascorbyl ester of a saturated fatty acid. The term “free” is intended to refer to the fatty acid that is not esterified. The term “not chemically bound” means that it is neither covalently nor ionically bonded to the ascorbic acid moiety. Hydrogen bonding or other interactions such as Van der Waals forces are not precluded.

The present invention provides a composition comprising an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative. The present invention provides a novel composition with the unexpected property of increasing the oxygen absorption rate over a composition containing the same amount of oxidizable ascorbic acid derivative and enzyme but without the fatty acid.

For the purposes of the present invention, the term “oxygen scavenging composition” will be employed to refer to the composition comprising an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative. In actual practice, this composition itself is not an oxygen scavenging composition. It does not actually react with oxygen unless moisture is present because the enzyme is inactive unless there is some moisture. The term “oxygen scavenging composition” will be employed to refer to both the inactive composition and to the moisture-activated composition. For practical purposes, sufficient moisture is available from ambient air or the air inside a package or container to effect oxygen scavenging with high activity. If insufficient moisture exists, a source of moisture can be added to the package or container in the form of a wet towel or the like.

Suitable oxidizable derivatives of ascorbic acid include but are not limited to ascorbate salts such as calcium ascorbate, magnesium ascorbate; and ascorbyl esters such as ascorbate palmitate, and other ascorbyl esters of saturated fatty acids. Preferred is calcium ascorbate or a combination of calcium ascorbate and ascorbate palmitate.

The free fatty acid is an unsaturated acid, which may be monounsaturated or polyunsaturated. Preferably it is a liquid at room temperature. Preferred free fatty acid is oleic acid. Suitable enzymes are multi-copper oxidases. The preferred enzyme is laccase.

Optionally the scavenging composition of the invention may contain additional materials such as a polymeric binder, a buffer, a hygroscopic agent and an inert filler.

Laccases (E.C. 1.10.3.2) are a class of multi-copper oxido-reductases (Systematic Name: Benzenediol:oxygen oxidoreductase) widely distributed in nature. This class of enzymes are capable of removing electrons from a wide range of oxidizable substrates via a four-electron reduction of molecular O₂ to form H₂O. Laccases are described in Reinhammar et al., Copper Proteins, T. G. Spiro, (Ed.), Wiley: New York, p 109-149. “Blue” copper-containing oxidases” (1981).

Numerous laccase variants are available from a multiplicity of sources both natural and synthetic. The operability of the present invention does not depend upon the particular laccase selected. However, different laccase variants exhibit different levels of activity and stability. Choice of a suitable laccase depends upon the particular requirements of any specific application of the invention.

The amount of laccase required in a package or container depends on package or container design parameters—such as internal volume, rate of O₂ ingress, desired rate of scavenging, desired residual O₂ concentration—and, enzyme related factors such as molecular weight, specific activity, and durability. Depending on those and other details, a range of enzyme concentration of 1-10,000 mg per mole of oxidizable ascorbic acid derivative is operable in the package or container hereof. Preferably the enzyme concentration is in the range of 1-200 μg/cm² within a coating.

The capacity of the O₂ scavenging system of the present invention is determined by the amount of reducing substrate available. Two moles of a two-electron reducing substrate are required to reduce one mole of molecular O₂ to H₂O. For example, about 3 g of sodium abscorbate (MW 198) are required to remove all the O₂ from 1 L of air at 25° C. In one embodiment wherein sodium abscorbate is used as the reductant, it is found that a loading of about 1-20 mg/cm² on a coated article is sufficient.

If the oxidizable ascorbic acid derivative is water soluble—such as calcium ascorbate—it can be do-dissolved in an same buffered aqueous solution with the enzyme followed by addition of an unsaturated fatty acid with sufficient agitation to create an emulsion.

If the oxidizable ascorbic acid derivative is not water soluble—such as ascorbyl palmitate—it can be dissolved in a suitable non-polar solvent (e.g., vegetable oil, polypropylene glycol) and mixed with the aqueous enzyme solution and the unsaturated fatty acid to form an emulsion. It may be desirable to also add an amphiphilic substance (e.g., lecithin) to help stabilize the emulsion.

It is important for achieving the best operability that the components of the composition be well-mixed. The enzyme, usually present in the resulting powder composition as small particles disposed upon the surface of the larger oxidizable ascorbic acid derivative particles, is well dispersed onto the surface of the oxidizable ascorbic acid derivative particles so that upon activation with moisture there is created an in situ micro-solution of enzyme on the oxidizable particle. Similarly, the free fatty acid is well-dispersed into the oxidizable particles. The degree of mixing and dispersion required will depend upon the requirements of the particular application. In general, however, a good guideline is simply the appearance of uniformity in the mixture, and good stability of the dispersions so obtained.

In a further embodiment, the oxygen scavenging composition further comprises monoethanolamine. In a further embodiment, micrometer sized iron particles are incorporated into the composition.

In general, it is found in the practice of the invention that advantageous compositions comprise, by weight, 30-40% of a binder polymer, 25-35% ascorbyl palmitate, 2-5% calcium ascorbate, 2-3% TiO2 or alumina, 0.5-1.5% laccase, 5-20% oleic acid or corn oil, and 0-4% monoethanolamine. Preferably monoethanolamine is present at ca.about 2% by weight, but in some applications the high degree of hygroscopicity of monoethanolamine is a disadvantage. In another embodiment is a method comprising contacting with molecular oxygen in the presence of moisture a composition comprising an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative, thereby causing at least a portion of the oxygen to be reduced by reaction with the oxidizable ascorbic acid derivative.

It is observed that the activity and stability of the enzyme is pH sensitive. It is preferred to maintain a neutral pH in the aqueous environment in which the oxygen reduction reaction takes place.

While the method may be performed with the oxygen scavenging composition in any form, it is advantageously applied when the oxygen scavenging composition is in the form of a coating or film on the surface of a shaped article. While the operability of the method is not dependent upon the shape of the coated article, preferably the article is a flat or contoured film or sheet that is readily inserted into or is itself a component of a package or container.

The coated article is prepared simply by contacting the oxygen scavenging composition with the surface of the article. The surface of the article may be treated with an adhesion promoter prior to application of the oxygen scavenging composition. Alternatively, the oxygen scavenging composition may further comprise an adhesion promoter admixed therewith. Coating or film forming may be accomplished by any convenient means in the art.

To form a coated article, the oxygen scavenging composition is typically combined with a liquid carrier to form a coating composition solution, suspension or ink that is applied to the surface of an article. The article may be printed, dip coated or painted with the coating composition. The technique of doctor-blading may be used to apply the coating composition to a flat surface. The term “ink” refers to a composition that comprises a colorant in combination with a solvent, an enzyme capable of using molecular O₂ as substrate, a suitable reductant, a polymeric binder and a thickening agent. The preferred solvent is water. Optionally, the ink may also include e.g., buffers, inert fillers, pigments and hygroscopic agents. The ink may be applied to a material by various methods, including spreading by wire-wound coating rod, rotary screen printing, flexographic printing, gravure printing and ink jet printing.

In one embodiment, the coated article hereof is a multilayer article including a layer upon which the oxygen scavenging composition is deposited and a so-called functional barrier layer that lies between the oxygen scavenging composition and the other contents of the package or container, such as a food item. The functional barrier layer is water vapor and oxygen permeable, but liquid water impermeable, and prevents the direct contact of the oxygen scavenging composition with the package or containerd goods.

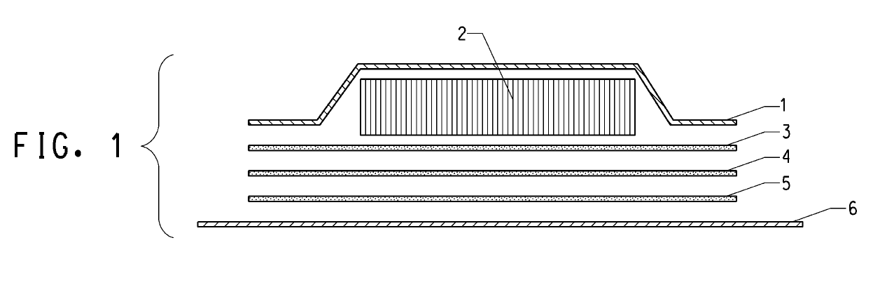

One embodiment is illustrated in FIG. 1. A multilayer label suitable for a container comprising a food product is shown that consists essentially of the following layers (wherein the order provided is from the side nearest the food product to the side I nearest the exterior of the package or container): functional barrier membrane (“1”), scavenger layer (“2”) containing the scavenging composition of the invention, adhesive layer (“3”), inter adhesive membrane (“4”), adhesive layer (“5”) and release backing (“6”) (FIG. 1).

Further provided according to the present invention is a package or container comprising contents and a sealing means, the contents comprising the composition. For the purpose of the present invention the term package or container refers to any shaped article that defines an interior space designed to hold an article or substance of any type and that upon sealing is substantially impermeable to O₂. Suitable containers include but are not limited to a pouch, bag, can, tank, barrel, silo, jar, box, envelope, bottle, or sealed wrapping. The contents can be solid, liquid, gaseous or mixtures thereof

The package or container of the invention comprises a sealing means when closed forms a sealed package. For the purpose of the present invention, the sealing means is any form of package or container closure that serves to separate the interior contents of the package or container from the exterior environment. The sealing means is preferably one that substantially prevents the diffusion of oxygen into the package or container. While the present invention requires the presence of a sealing means, there is no requirement that the package or container be sealed therewith, although it is preferred.

The composition of the present invention, particularly when in the form of a coating or film may further comprise a polymeric binder. Suitable binders include but are not limited to aqueously insoluble polymers such as neoprene, styrene butadiene rubber, olefin ionomers, vinyl acetate ethylene copolymer and natural rubber; or aqueously soluble polymers such as poly vinyl alcohol, carboxymethyl cellulose, hydroxypropyl methyl cellulose and soy protein. Aqueously soluble polymers are preferred simply for the ease of combining with a soluble enzyme and oxidizable ascorbic acid derivative in emulsion with a fatty acid.

Carboxymethyl cellulose or hydroxypropyl methyl cellulose are most desirable for use as binders, since they permit formation of stable (e.g., 1-30 days), high viscosity solutions at low levels of enzyme (e.g., about 0.02 to 0.2 weight %) and tolerate the required amount of ascorbate.

The composition may further comprise up to 50% by weight of a hygroscopic agent may optionally be included within the O₂ scavenging composition for enhancing the activation of the oxygen scavenging reaction. Suitable hygroscopic agents include but are not limited to fructose, silica gel, or polyvinyl alcohol. It is found in the practice of the invention that ascorbate salts are normally sufficiently hygroscopic that additional hygroscopic agents are not required for acceptable activation.

Polymers suitable for use in forming the so-called functional barrier need to be oxygen and moisture permeable. Suitable polymers included but are not limited to polyacrylonitrile, polymethacrylonitrile, polyvinylidene chloride, polyethylene terephthalate, Nylon 6®, polyvinyl chloride, cellulose acetate, cellulose acetate butyrate, cellulose diacetate, Neoprene®, poly 4-methyl pentene-1 and poly dimethyl siloxane.

There is no limitation on the type of article to the surface of which the O₂ scavenging composition can be applied. Suitable materials include wood pulp filter paper, glass fiber filter paper, paperboard, woven and nonwoven fabrics, polymer films and paper such as label stock. It is found in the practice of the present invention that coatings on paper exhibit greater O₂ scavenging rates, although wetting of the paper can be a problem in some circumstances.

The operability of the present invention is not limited by the method of application of the oxygen scavenging composition to the surface. Suitable methods include but are not limited to spreading by wire-wound coating rod, rotary screen printing, flexographic printing, spraying, blotting, dipping, coating and ink jetting, and any other method known in the art.

The O₂ scavenging composition can be applied to a surface in one or more homogeneous layers, wherein the enzyme, oxidizable ascorbic acid derivative, unsaturated free fatty acid and any other optional components are first prepared as an intimate mixture. This mixture can take the form of a solution, dispersion, or emulsion, including pastes. In one embodiment, the oxygen scavenging composition is formulated for gravure printing. In another embodiment, the composition is an ink comprising the oxygen scavenging composition formulated for ink jet printing.

Alternatively, several of the components of the oxygen scavenging composition can be combined in situ in the package where it is to be used. For example, the enzyme can be printed onto a label while a mixture of the oxidizable ascorbic acid derivative and free fatty acid is printed onto a functional barrier film. When the package is assembled, the functional barrier film may be applied to the label surface, thereby combining the enzyme and the oxidizable ascorbic acid derivative. This can be a quite useful method when it is desired to avoid activation of the oxygen scavenger until the moment of package assembly without having to use expensive handling arrangements to keep the composition dry.

The oxygen scavenging compositions can be stored for months at a time in a dried or frozen state. This permits preservation of the enzymatic activity and prevention of premature activation of the system, prior to the commencement of O₂ scavenging within a sealed container. The O₂ scavenging compositions may be stored prior to their application onto a surface, following their application onto a surface, or as a complete O₂ scavenging system that is present in a sealed container.

Many approaches can be used to construct packaging for foodstuffs and other items using the oxygen scavenging composition. When the oxygen scavenging composition is incorporated within the wall of a sealed container, the container wall may be a layered construction (e.g., co-extruded, extrusion-coated, coated, laminated) that is optionally bonded with adhesives. Such a layered construction can be prepared by co-extrusion, extrusion-coating, solution or dispersion coating, lamination, and other means such as are known in the art. In one embodiment, there is direct contact between the packaged item—such as food—and a functional barrier layer the function of which is to prevent direct contact between the oxygen scavenging composition and the contents of the container while permitting diffusion of O₂ from the headspace of the sealed container through the functional barrier so that it may react with the O₂ scavenging composition. The functional barrier may be separated from the exterior layer of the sealed container by any number of layers. There is, no limitation to shape, degree of flexibility, thickness, or number of layers in the final construction.

In one embodiment, a solution comprising the oxygen scavenging composition is applied by a gravure roller and the coated paperboard is then dried in a stream of nitrogen. One side of the resultant paperboard is extrusion coated with low-density polyethylene (“LDPE”, a suitable functional barrier), while the reverse face of the paperboard is coated with a high O₂ barrier layer (e.g., ethylene vinyl alcohol copolymer), combined with tie layers and other polymer layers as desired to produce a multilayer packaging material. The LDPE layer is ultimately in contact with the liquid contents of the sealed container, while the O₂ barrier layer is on the outside of the container facing the atmosphere.

In another embodiment, the O₂ scavenging composition can be combined with a carrier polymer matrix and applied to a foil laminate surface. The polymer matrix may be derived from a variety of polymers and formulated as a dispersion, latex, emulsion, plastisol, dry blend, or solution. After the matrix is applied, it is dried to stabilize the reducing activity and a final lamination of LDPE as the functional barrier, construction of a package or container by this method would permit production of a laminated material useful in forming e.g., pouches or beverage boxes. Similarly, the O₂ scavenging composition can be combined with a carrier polymer matrix and applied to multicoated paperboard, then coated with a layer of polymer (e.g., LDPE). Such a material would also be useful in making containers for juices and other liquids (e.g., a jug, carton).

In another embodiment, the oxygen scavenging composition will be incorporated into an insert (e.g., a pouch, sachet, envelope, canister, vial, adhesive patch, label, gasket, lid, cap, card, liner, etc.) that is then placed within the container.

In one embodiment, a liquid or solid oxygen scavenging composition may be applied to a mat, card or disk composed of fibers, such that the composition is contained within the interstices of the fibers; a sponge or polymeric foam, wherein the composition is contained within the pores of the foam; a granular or particulate matrix, wherein the matrix can be derived from natural polymers (e.g., cellulose), synthetic polymers, clays, high surface area metal oxide particles, or combinations thereof. After application the composition may optionally be dried or frozen to preserve activity. Subsequently, the wetted, dried, or frozen fibrous material, sponge or matrix may then be enclosed within an insert. The insert can be of any configuration (e.g., a pouch, sachet or envelope made of an O₂ permeable polymeric sheet or film; a canister or vial). Following enclosure within the container and sealing thereof, the O₂ scavenging system can be frozen to preserve activity.

In some embodiments, the oxygen scavenging composition may be disposed on a patch or label that is physically attached to the container and prevents easy removal from the container by the consumer. A label or patch is expected to be particularly suitable for use in containers comprising a non-liquid food product (e.g., fresh pasta, meat). The oxygen scavenging composition can be coated or adsorbed onto a surface, and can be used moist, or can be dried or frozen to preserve activity. The mat, card disk, sponge, foam, or matrix is then affixed to the container with a functional barrier that provides a means for O₂ transport. The functional barrier can be of any configuration, provided that it enables isolation of the O₂ scavenging system from the contents of the container. For example, the functional barrier can be, but is not limited to: an inherently gas-permeable polymer; a porous material (e.g., spun-bonded polymer or open cell foam); or, a solid material rendered permeable by perforations. The complete O₂ scavenging system can be placed, positioned, or affixed anywhere within the container to be sealed.

When the oxgyen scavenging composition is used in connection with liquid contents, isolation of the O₂ scavenging composition can be achieved by placing the composition behind a functional barrier that is composed of a polymer film that is permeable to O₂ and water vapor, but not liquid water. Alternatively, the O₂ scavenging system can be applied to one side of a patch or label. Upon drying of the O₂ scavenging composition, the coated surface of the patch or label can be applied to the inside of a container, or a film used to seal a container. Or, in another embodiment, the coated surface of the patch or label can be covered with an O₂ permeable, thin film and then the multilayered structure can be affixed to the container.

In other instances, it will be desirable for the patch or label to be affixed to the outside of the container to be sealed. In this case, the patch or label will be applied over a zone of perforations or an alternative site providing a means for O₂ transport from the interior of the container to the exteriorly affixed patch or label and its O₂ scavenging system.

In another embodiment of the invention, the O₂ scavenging system can be formulated within a polymer matrix. The formulation may be a dispersion, latex, emulsion, plastisol, dry blend or solution. The components can be formulated within the polymer by any method known in the art that does not degrade the components of the O₂ scavenging system and is inert with respect to the contents of the container. The formulation so prepared can be deposited onto the interior of the container to be sealed as a patch, gasket, coating, or film, for example. The patch, gasket, coating or film may inself embody a functional barrier, or may be covered by a separately applied functional barrier by an additional coating or lamination step. In one embodiment, the formulation can be applied to shrink wrap film and used to wrap containers.

In another embodiment, the oxygen scavenging composition can be incorporated into container closures such as gaskets, lid liners, caps, corks, septa, or plugs. In one embodiment, a polymeric formulation is made into sheet form and gaskets are stamped out therefrom. In another embodiment, the oxygen scavenging composition is combined with a carrier polymer matrix that is deposited directly on caps or closures to form gaskets or lid liners. In another embodiment, the O₂ scavenging composition can be incorporated directly into the matrix of a cork or plug or the composition can be contained within a reservoir inside the cork or plug.

The oxygen scavenging composition is well-suited for use in food and beverage packaging. Embodiments comprise so-called food-safe components such as laccase, calcium ascorbate, and oleic acid. Other applications are also encompassed in the present invention where an altered gaseous environment is desirable. This includes use for maintaining anaerobic bacterial cultures; for preservation of electronic components; for preservation of cosmetics and personal care products; for inerting aircraft fuel tanks (to prevent flammable fuel/air vapors in fuel tanks); and in packaging of specific pharmaceutical compositions.

The invention is further described but not limited in the following specific embodiments.

### EXAMPLES

General Procedure for Examples

Tests of oxygen scavenging of dried coating formulations and coated films were conducted in a 150 cc pressure vessels (model CG-1880-41-150 Chemglass) with #25 internal thread and drilled caps in which an S-101 oxygen sensor (Qubit Systems) was installed. Humidity was supplied in each vessel by means of moist paper towel or wet cotton. All scavenging experiments were conducted at 23° C. Sensor readings were monitored by a LabPro interface (Vernier) connected to a computer equipped with an uninterruptible power supply and running Logger Pro software (Qubit Systems).

The O₂ sensor was permanently attached to an amplifier having a gain control with an output range of 0 to 4.5 volts. The output of the sensor was linear across all oxygen partial pressure ranges. For use in the range incorporating oxygen at atmospheric pressure, the sensor was calibrated by exposing the sensor to standard air (20.95% O₂) and setting the output voltage to 2.20 volts.

Unless otherwise noted, the containers employed were 100 ml screw top glass jars. Stirring was effected using a magnetic stirring bar and was generally performed for 15 minutes.

The film employed was type 1000A FEP®100 from the DuPont Company.

The drying oven was a vacuum oven VWR model 1430, equipped with a nitrogen purge.

Calcium ascorbate/laccase powder was produced by spray drying a 25% by weight solution of calcium ascorbate in water containing 0.25% laccase enzyme. Spray drying was done in a 3 ft diameter, 15 ft³ volume, pilot spray dryer. The dryer was supplied with drying air heated to 228° C. A peristaltic pump was used to meter feed solutions to the spray-drying nozzle. A Spraying Systems SU4, dual fluid nozzle supplied with 30 psi N₂ was used to spray slurries into the volume of the dryer. 75° C. aerosol discharged the dryer to an 8 ft² bag filter where entrained solids were disengaged from the spent drying gas.

Results were determined in terms of the remaining oxygen concentration after 60 hours, designated [O₂]60. Results for all the examples are summarized in Table 1.

TABLE 1

| | | | | | | Fatty | |
|---|---|---|---|---|---|---|---|
| | Substrate | | | | Fatty | Acid | [O₂]₆₀ |
| Example | Type | % | Laccase | Filler | Acid | (%) | (%) |
| CE A | CA | 0 | 0 | TiO2 | oleic | 20 | 22 |
| CE B | CA | 25 | yes | none | none | | 18 |
| 1 | CA | 15 | yes | nano | corn | 20 | 12 |
| | | | | TiO2 | | | |
| 2 | CA | 15 | yes | alumina | oleic | 20 | 12 |
| 3 | CA | 15 | yes | nano | oleic | 20 | 12 |
| | | | | TiO2 | | | |
| 4 | CA | 15 | yes | P25 | oleic | 20 | 12 |
| | | | | TiO2 | | | |
| 5 | CA | 35 | yes | none | oleic | 30 | 9 |
| CE C | CA | 35 | yes | none | none | | 15 |
| CE D | AP | 15 | none | TiO2 | oleic | 20 | 21 |
| CE E | AP | 15 | yes | none | none | | 20 |
| CE F | CA | 15 | yes | TiO2 | none | | 12 |
| 6 | CA | 15 | yes | none | oleic | 20 | 12 |
| 7 | AP/CA | 30/5 | yes | P25 | oleic | 15 | 16 |
| | | | | TiO₂ | | | |
| 8 | AP/CA | 30/5 | yes | TiO₂ | oleic | 15 | 12 |

### Comparative Example A

### 72% CAP-482-0.5/3% TiO2/20% OA/5% Triacetin

In a first container, 7.2 g. of cellulose acetate propionate (CAP 482-0.5, Eastman Chemical) was dissolved in 15 g of ethyl acetate and 0.5 g triacetin (Eastman Chemical) was added. The resulting solution was stirred until it became clear. In a second container, 2 g of Oleic acid, 10 g ethyl alcohol and 0.3 g of TiO2 particles (Hombitec RM 130F).were combined and stirred until the TiO2 particles were uniformly dispersed. The contents of the second container were then added to the contents of the first container and the mixture was stirred to form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Comparative Example B

### 70% CAP 482-0.5/25.0% Calcium Ascorbate-Laccase/5% Triacetin

7 g. of CAP 482-0.5 was dissolved in 15 g of ethyl acetate and 0.5 g Triacetin was added to the prepared solution. The resulting solution was stirred until it became clear. 2.5 g of calcium ascorbate-Laccase powder and 10 g ethyl alcohol were added to the stirred solution, and the resulting combination was further stirred. 1 g of coating was prepared as in Comparative Example A, and tested.

### Example 1

### 57% CAP 482-0.5./15.0% Calcium Ascorbate-Laccase/3% TiO2/20%. Corn oil/5% Triacetin

In a container-5.7 g. of CAP 482-0.5 was dissolved in 20 g. of ethyl acetate and 0.5 g Triacetin was added. The resulting solution was stirred until clear. To the prepared solution were added 2 g of corn oil, 3 g. of ethyl acetate, 1.5 g. of calcium ascorbate-Laccase and 0.3 g of TiO2 particles (Hombitec RM 130F). The resulting combination was stirred until the TiO2 particles were uniformly dispersed. 1 g of coating was prepared as in Comparative Example A and tested.

### Example 2

### 57% CAP 482-0.5/15.0% Calcium Ascorbate-Laccase/3% Alumina/20%. OA/5% Triacetin

In a first container, 5.7 g. of CAP 482-0.5 was dissolved in 15 g of ethyl acetate and 0.5 g Triacetin was added. The resulting solution was stirred until it became clear. In a second container, 2 g of Oleic acid, 10 g ethyl alcohol, 1.5 g Calcium ascorbate-Lacase powder and 0.3 g of alumina particles (AL205D) were combined and stirred until the alumina particles were uniformly dispersed. The contents of the second container were then added to the contents of the first container and the mixture was stirred to form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Example 3

### 57% CAP-482-0.5/7.5% Calcium Ascorbate-Lacase/7.5% Ascorbyl Palmitate/3% TiO2//20%. OA/5% Triacetin

In a first container, 5.7 g. CAP 482-0.5 was dissolved in 15 g of ethyl acetate and 0.5 g Triacetin was added. The resulting solution was stirred until it became clear. In a second container, 0.75 g of Ascorbyl palmitate was dissolved in 10 g ethyl alcohol at 40 C. 0.75 g calcium ascorbate, 2 g Oleic acid and 0.3 g of TiO₂ particles (MT 100S) were added to the prepared solution and stirred until the TiO₂ particles were uniformly dispersed. The contents of the second container were then added to the contents of the first container and the mixture was stirred to form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Example 4

### 57% CAP-482-0.5/15% Calcium Ascorbate-Laccase/3% TiO2//20%. OA/5% Triacetin

In a first container, 5.7 g. CAP 482-0.5 was dissolved in 15 g of ethyl acetate and 0.5 g Triacetin was added. The resulting solution was stirred until it became clear. In a second container, 2 g of Oleic acid, 10 g ethyl alcohol, 1.5 g Calcium ascorbate-Lacase powder and 0.3 g of TiO2 particles (Aeroxide P25 from Degussa Corp) were combined and stirred until the alumina particles were uniformly dispersed. The contents of the second container were then added to the contents of the first container and the mixture was stirred to form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Example 5

### 35% CAP 482-0.5/35% Calcium Ascorbate-Laccase/30%. OA

In a first container, 3.5 g. CAP 482-0.5 was dissolved in 11 g of ethyl acetate. The resulting solution was stirred until it became clear. In a second container, 3 g of Oleic acid and 3.5 g. of calcium ascorbate-Laccase were combined in 10 g ethanol. The resulting mixture was added to the first container while stirring tor form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Comparative Example C

### 45% CAP 482-0.5/35% Calcium Ascorbate-Laccase/10% Triacetin/10% Cymel 385

In a first container, 4.5 g. CAP 482-0.5 was dissolved in 15 g of ethyl acetate and 1.0 g Triacetin was added. The resulting solution was stirred until it became clear. In a second container, 3.5 g. of calcium ascorbate-laccase powder and 1.0 g of Cymel 385 was dissolved in 10 g. of ethanol. The contents of the second container were then added to the contents of the first container and the mixture was stirred to form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Comparative Example D

### 57% CAP 482-0.5/15% Ascorbyl Palmitate/20% OA/3% TiO2/5% Triacetin

In a first container, 5.7 g. CAP 482-0.5 was dissolved in 15 g of ethyl acetate and 0.5.g Triacetin was added. The resulting solution was stirred until it became clear. In a second container, 1.5 g of ascorbyl palmitate was dissolved in 20 g. of ethanol at 40 degrees C. 2.0 g. of oleic acid followed by 0.3 g of TiO2 (Hombitec RM 130F) were added to the prepared solution. The resulting mixture was stirred until the TiO₂ particles were uniformly dispersed. The contents of the second container were then added to the contents of the first container and the mixture was stirred to form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Comparative Example E

### 78% CAP 482-0.5/15% Ascorbyl Palmitate/5% Triacetin/2% Laccase Enzyme

In a first container, 7.8 g. CAP 482-0.5 was dissolved in 25 g of ethyl acetate. The resulting solution was stirred until it became clear. In a second container, 1.5 g of ascorbyl palmitate was dissolved in 10 g. of ethanol at 40 degrees C. The contents of the second container were then added to the first container, and the resulting mixture was stirred. 0.5 g. of Triacetin and 2.3 g. laccase enzyme solution (containing 0.2 g. active laccase.) were added with further stirring. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Comparative Example F

### 82% CAP 482-0.5/15% Calcium Ascorbate-Laccase/3% TiO2

In a first container, 8.2 g. CAP 482-0.5 was dissolved in 20 g of ethyl acetate. The resulting solution was stirred until it became clear. In a second container, 10 g of ethyl alcohol and 1.5 g of calcium ascorbate-laccase powder were combined with stirring. The contents of the second container were than added to the first container. To the mixture so formed, 0.3 g. TiO2 (Hombitec RM 130F) was added and the resulting mixture was stirred until the TiO₂ particles were uniformly dispersed, thereby forming a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Example 6

### 65% CAP 482-0.5/15% Calcium Ascorbate-Laccase/20%. OA

In a container, 6.5 g. of CAP 482-0.5 was dissolved in 25 g of ethyl acetate. The resulting solution was stirred until it became clear. 1.5 g calcium ascorbate-laccase powder was added to the stirred solution, and the resulting mixture was further stirred. 2.0 g of oleic acid was then added with further stirring to form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Example 6

### 40% CAP 482-0.5/30% Ascorbyl Palmitate/5% Calcium Ascorbate-Laccase/15% OA/3% TiO2/5% Triacetin/2% La

In a first container, 4.0 g. of CAP was dissolved in 25 g of ethyl acetate. The resulting solution was stirred until it became clear. In a second container, 3.0 g of ascorbyl palmitate was dissolved in 15 g. of ethanol at 40 degrees C. 0.5 g of calcium ascorbate-laccase was added to the resulting solution. The contents of the second container were then added to the first container with stirring. 1.5 g. of oleic acid followed by 0.3 g. of Aeroxide P25 TiO₂ were added and the mixture stirred until the TiO₂ particles were uniformly dispersed. 0.5 g. of Tricacetin and 2.3 g. laccase enzyme solution (0.2 g. active laccase.) were then added followed by further stirring for form a coating formulation. The prepared coating formulation was applied to a polytetrafluoroethylene film using a #90 wire rod. The coated film was then transferred to an oven and dried at 50° C. under nitrogen. 1 g of the dried coating was removed from the FEP film and transferred to the humid pressure vessel and sealed, as described supra.

### Example 8

### 21.6% CAP/13.5% Elvaloy/30% Asc Palm/5% CaAsc/3% TiO2/15% OA/0.5% Laccas/11.4% MEA (Solids Basis)

In a first container, 2.16 g. CAP 482-0.5 add 0.6 g. Laccase Enzyme solution were combined, stirred, and dried overnight at 50 C with N₂ purge. The dried powder was then dissolved in 15 g ethyl acetate. In a second container, 1.35 g. Elvaloy® 742 available from the DuPont Company was dissolved in 10 g. THF. 3.0 g. ascorbyl palmitate, 3 g. THF, and 5 g. ethanol were added to the prepared solution while stirring. The mixture was heated to just below 40° C. until a clear solution was formed. The solution so formed was removed from the heat and 1.5 g. oleic acid was added, followed by the contents of the first container, while stirring. 0.3 g. Hombitec RM130F TiO2 was added, and the resulting mixture stirred until the TiO₂ was well-dispersed. Separately, a solution of 0.51 g of calcium ascorbate in 1.14 g of monoethanolamine was prepared and added to the mixture in the second container. Viscosity was adjusted with a solvent mixture prepared ahead of time consisting of 26 g THF/32 g. ethyl acetate/10 g. ethanol.

Films were prepared as described supra.

What is claimed is:

1. A composition comprising an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative.

2. The composition of claim 1 wherein the oxidizable ascorbic acid derivative is calcium ascorbate.

3. The composition of claim 2 further comprising ascorbyl palmitate.

4. The composition of claim 1 wherein the multi-copper oxidase enzyme is laccase.

5. The composition of claim 1 where in the composition, the free fatty acid is corn oil.

6. The composition of claim 1 where in the composition, the free fatty acid is oleic acid.

7. The composition of claim 1 further comprising monoethanolamine.

8. The composition of claim 1 wherein the composition further comprises a transition metal oxide filler particle.

9. The composition of claim 8 where in the composition, the transition metal oxide is titanium dioxide or alumina.

10. A method comprising a coated film, the coated film comprising exposing to oxygen in the presence of moisture a composition comprising an oxidizable ascorbic acid derivative, a multi-copper oxidase enzyme, and a free fatty acid wherein the enzyme is disposed upon the surface of the oxidizable ascorbic acid derivative, and wherein the free fatty acid is intermixed with the oxidizable ascorbic acid derivative.

11. The method of claim 10 wherein the exposing is performed within a sealed package or container.

12. A coating composition comprising the composition of claim 1 dissolved or suspended in a carrier liquid forming a coated film.

13. An article comprising a surface whereupon is disposed the coating of claim 12.

14. A package or container comprising contents and a sealing means, the contents comprising the composition of claim 1.

15. A package comprising the coated film of claim 12.
